(19) United States
(12) Patent Application Publication
(10) Pub. No.: US 2024/0050393 A1
(43) Pub. Date: Feb. 15, 2024

(54) COMPOSITIONS AND USES THEREOF FOR TREATING OR PREVENTING VIRAL INFECTIONS

(71) Applicant: GILVERDI - HEALTH AND BEAUTY LTD, Jerusalem (IL)

(72) Inventor: Vered GILAD, Beit Shemesh (IL)

(21) Appl. No.: 18/258,919

(22) PCT Filed: Dec. 24, 2021
(86) PCT No.: PCT/IL2021/051533

§ 371 Date: Jun. 22, 2023

(30) Foreign Application Priority Data
Dec. 24, 2020 (IL) 279765

(51) Int. Cl. A61K 31/245; A61K 31/015; A61K 31/05; A61K 45/06; A23L 33/00; A61P 31/14

(52) U.S. Cl. CPC A61K 31/245; A61K 31/015; A61K 31/05; A61K 45/06; A23L 33/00; A61P 31/14

(57) ABSTRACT

The present invention relates to compositions comprising one or more plant- or fungus-derived compounds and their use for treating or preventing viral respiratory infections (VRIs), such as COVID-19.

### FIELD OF THE INVENTION

The present invention relates to compositions and methods useful for treating or preventing viral infections. More particularly, the invention relates to compositions comprising one or more plant- or fungus-derived compounds and their use for treating or preventing viral respiratory infections (VRIs).

### BACKGROUND OF THE INVENTION

A viral respiratory infection refers to an infection which is caused by a virus that specifically affects the upper or lower respiratory tracts or both. Respiratory viruses are the most frequent causative agents of disease in humans, with a significant impact on morbidity and mortality worldwide. Several of these viruses circulate commonly in all age groups and are adapted to efficient person-to-person transmission. Moreover, as these viruses frequently mutate, most of the population has little if any immunity to new strains. In the last three centuries, at least 12 pandemics have been caused by respiratory viruses, such as H1N1 (“Spanish flu”), and coronaviruses (severe acute respiratory syndrome or SARS, Middle East respiratory syndrome or MERS). The 2019 severe acute respiratory syndrome coronavirus 2 (SARS-CoV-2) is a newly emerging respiratory illness. The epidemic started in December 2019 in Wuhan, China, and has rapidly spread throughout China and the world and is now a global pandemic. As of Nov. 15, 2020, more than 50 million cases have been confirmed, and more than 1.07 million deaths were attributed to coronavirus disease (COVID-19). There are currently no approved vaccines available for the prevention of SARS-CoV-2.

It is therefore an object of the present invention to provide a composition comprising one or more plant- or fungus-derived compounds for treating or preventing viral respiratory infections.

It is another object of the invention to provide a dietary supplement useful for treating or preventing viral respiratory infections.

It is yet another object of the invention to provide a method of treating or preventing viral respiratory infection.

Other objects and advantages of the invention will become apparent as the description proceeds.

### SUMMARY OF THE INVENTION

In one aspect, the present invention provides a composition comprising one or more plant-derived or fungus-derived compounds selected from D-Limonene, menthol, linalyl anthranilate, zingiberene, p-menthone, linalool, p-menthan-3-one, 1,8-cineole, tumerone, Ar-tumerone, (−)-β-sesquiphellandrene, α-curcurmene, (1S)-(−)-camphor, curlone, 2-β-pinene, β-bisabolene, α-pinene, γ-terpinene, camphene, α-farnesene, terpinene-4-ol, γ-muurolene, trans-caryophyllene, (L)-α-terpineol, (1R)-p-mentha-3-ene, β-ocimene, β-myrcene, (±)-lavandulol acetate, o-cymol, (+)(−)trans-carane, ocimene, β-citral and alloaromadendrene, or any combination thereof.

In one embodiment of the invention, the one or more plant-derived or fungus derived compounds are present in the composition at concentration ranges according to the following table:

| Name of compound | Concentration range (ppm) |
|---|---|
| D-Limonene | 6828-8346 |
| Menthol | 4946-6046 |
| Linalyl anthranilate | 3898-4766 |
| (linalyl 2-aminobenzoate) | |
| Zingiberene | 3571-4365 |
| p-Menthone (trans-menthone) | 2847-3481 |
| Linalool | 2592-3168 |
| p-Menthan-3-one | 2199-2689 |
| 1,8-Cineole | 2044-2500 |
| Tumerone (α-turmerone) | 1998-2442 |
| Ar-tumerone | 1937-2369 |
| (−)-β-Sesquiphellandrene | 1586-1940 |
| α-Curcurmene | 1263-1545 |
| (1S)-(−)-Camphor | 1095-1339 |
| Curlone | 1080-1322 |
| 2-β-Pinene | 1071-1309 |
| β-Bisabolene | 874-1070 |
| α-Pinene | 856-1048 |
| γ-Terpinene | 693-847 |
| Camphene | 683-835 |
| α-Farnesene | 655-801 |
| Terpinene-4-ol | 453-555 |
| γ-Muurolene | 440-538 |
| Trans-Caryophyllene (β-caryophyllene) | 360-440 |
| (L)-α-Terpineol | 355-435 |
| (1R)-p-Mentha-3-ene | 234-286 |
| β-Ocimene (ocimene (E)) | 215-263 |
| β-Myrcene | 201-247 |
| (±)-Lavandulol acetate (lavandulyl acetate) | 187-229 |
| o-Cymol (o-cymene) | 178-218 |
| (+)(−)Trans-Carane | 140-172 |
| Ocimene | 117-143 |
| E-Citral (β-citral) | 102-126 |
| Alloaromadendrene | 89-109 |

In a specific embodiment of the invention, the composition comprises D-Limonene, menthol, and linalyl anthranilate, wherein D-Limonene, menthol, and linalyl anthranilate are present in the composition at concentration ranges according to the following table:

| Name of compound | Concentration range (ppm) |
|---|---|
| D-Limonene | 6828-8346 |
| Menthol | 4946-6046 |
| Linalyl anthranilate | 3898-4766 |

In another specific embodiment of the invention, the composition comprises the compounds listed in the following table at the specified concentrations:

| Name of compound | Concentration (ppm) |
|---|---|
| D-Limonene | 7587 |
| Menthol | 5496 |
| Linalyl anthranilate | 4332 |
| (linalyl 2-aminobenzoate) | |
| Zingiberene | 3968 |
| p-Menthone (trans-menthone) | 3164 |
| Linalool | 2880 |
| p-Menthan-3-one | 2444 |
| 1,8-Cineole | 2272 |
| Tumerone (α-turmerone) | 2220 |
| Ar-tumerone | 2153 |
| (−)-β-Sesquiphellandrene | 1763 |
| α-Curcurmene | 1404 |
| (1S)-(−)-Camphor | 1217 |
| Curlone | 1201 |
| 2-β-Pinene | 1190 |
| β-Bisabolene | 972 |
| α-Pinene | 952 |
| γ-Terpinene | 770 |
| Camphene | 759 |
| α-Farnesene | 728 |
| Terpinene-4-ol | 504 |
| γ-Muurolene | 489 |
| Trans-Caryophyllene (β-caryophyllene) | 400 |
| (L)-α-Terpineol | 395 |
| (1R)-p-Mentha-3-ene | 260 |
| β-Ocimene (ocimene (E)) | 239 |
| β-Myrcene | 224 |
| (±)-Lavandulol acetate (lavandulyl acetate) | 208 |
| o-Cymol (o-cymene) | 198 |
| (+)(−)Trans-Carane | 156 |
| Ocimene | 130 |
| E-Citral (β-citral) | 114 |
| Alloaromadendrene | 99 |

In another embodiment of the invention, the one or more compounds are derived from one or more plants and/or edible fungi selected from black elderberry, turmeric, cat's claw, female ginseng, fennel, licorice, bitter orange, purslane, greater galangal, myrrh, saffron, flowering thick-stemmed sugar cane, ginger, lavender, mint, rosemary, lemon, reishi, Agaricus hortensis, or any combination thereof.

In yet another embodiment of the invention, the composition comprises at least one extract or essential oil derived from one or more of said plants and/or edible fungi.

According to a specific embodiment of the invention, the composition comprises extracts derived from cat's claw, reishi, black elderberry, turmeric, Agaricus hortensis, female ginseng, fennel, licorice, bitter orange, greater galangal, myrrh, saffron, purslane and flowering thick-stemmed sugar cane, and essential oils derived from turmeric, mint, ginger, lavender, lemon and rosemary.

According to a further embodiment of the invention, the composition is in the form of a tablet, pill, capsule, cachet, lozenge, granule, semisolid, powder, sustained release formulation, solution, suspension, emulsion, elixir, or aerosol.

In yet a further embodiment of the invention, the composition is formulated as a dietary supplement or as a nutritional supplement.

In another aspect, the present invention provides a composition as described above for use in treating or preventing a viral respiratory infection or disease, disorder or condition caused by a viral respiratory infection.

In a further aspect, there is provided a composition as described above for use in alleviating symptoms of a disease, disorder or condition caused by a viral respiratory infection.

In one embodiment of the invention, the composition is administered concurrently with a vaccination and/or an anti-viral medication.

In another embodiment of the invention, the composition is administered orally to a subject.

In a specific embodiment of the invention, the composition is administered to the subject by adding said composition to a drink, solid food or semi-solid food to be consumed by the subject.

In another specific embodiment of the invention, the composition is in liquid form and is administered to the subject at an amount of between 1 to 100 drops per day.

In yet another specific embodiment of the invention, preventing a viral respiratory infection or disease, disorder or condition caused by a viral respiratory infection comprises administering the composition at an amount of 4-15 drops per day.

In a further specific embodiment of the invention, treating a viral respiratory infection or a disease, disorder or condition caused by a viral respiratory infection or alleviating symptoms of a disease, disorder or condition caused by a viral respiratory infection comprises administering the composition at an amount of 20-100 drops per day.

In yet a further specific embodiment of the invention, treating a viral respiratory infection or a disease, disorder or condition caused by a viral respiratory infection or alleviating symptoms of a disease, disorder or condition caused by a viral respiratory infection comprises administering the composition at an amount of 70-100 drops per day during the first 1-3 days after the diagnosis of the disease, disorder or condition was made or after the onset of the symptoms, followed by an amount of 20-40 drops per day for additional 1-14 days, or until the patient has recovered, or until the symptoms have subsided or completely disappeared.

In yet a further aspect, the present invention provides a method of treating or preventing a viral respiratory infection or disease, disorder or condition caused by a viral respiratory infection in a subject, the method comprising administering a composition as disclosed herein above to the subject.

In another aspect of the invention, there is provided a method of alleviating symptoms of a disease, disorder or condition caused by a viral respiratory infection in a subject, comprising administering a composition as disclosed herein above to the subject.

In one embodiment of the invention, the composition is administered to the subject concurrently with a vaccination and/or an anti-viral medication.

In another embodiment of the invention, the composition is administered orally to the subject.

In a further embodiment of the invention, the composition is administered to the subject by adding said composition to a drink, solid food or semi-solid food to be consumed by the subject.

In yet a further embodiment of the invention, the composition is in liquid form and is administered to the subject at an amount of between 1 to 100 drops per day.

According to a specific embodiment, the preventing a viral respiratory infection or disease, disorder or condition caused by a viral respiratory infection comprises administering the composition to the subject at an amount of 4-15 drops per day.

According to another specific embodiment of the invention, the treating a viral respiratory infection or a disease, disorder or condition caused by a viral respiratory infection or alleviating symptoms of a disease, disorder or condition caused by a viral respiratory infection in the subject comprises administering the composition to the subject at an amount of 20-100 drops per day.

According to a further embodiment of the invention, the treating a viral respiratory infection or a disease, disorder or condition caused by a viral respiratory infection or alleviating symptoms of a disease, disorder or condition caused by a viral respiratory infection in the subject comprises administering the composition to the subject at an amount of 70-100 drops per day during the first 1-3 days after the diagnosis of the disease, disorder or condition was made or after the onset of the symptoms, followed by an amount of 20-40 drops per day for additional 1-14 days, or until the patient has recovered, or until the symptoms have subsided or completely disappeared.

### BRIEF DESCRIPTION OF THE DRAWINGS

FIG. 1 shows cytotoxicity levels as relative absorbance (RA) of MTT assay in cells infected with SARS-CoV2 that were pretreated with a compound according to one embodiment of the invention (CoV2+T) compared to infected untreated cells (CoV2) and uninfected negative control cells (NC).

### DETAILED DESCRIPTION OF THE INVENTION

The compositions and methods according to the invention are provided for the treatment and prevention of viral infections. Viral infections, and specifically respiratory viral infections, are highly infectious and are the cause of a series of pandemic outbreaks throughout history, including the recent 2019 coronavirus disease (COVID-19). Furthermore, the development of vaccines against respiratory viral infections is a relatively slow process compared to the rate at which the disease is spread in the population. Moreover, as these viruses often mutate, additional outbreaks of the newly mutated viruses are expected to reoccur every few months or years. Considering that the previously developed vaccines (if any) would be less efficient or even completely useless against the mutated virus, predictions of how the virus is likely to be mutated and continuous efforts to constantly develop new vaccines are required in order to efficiently prevent future infections. However, these efforts often result in a misprediction of the characteristics of the mutated virus and consequently failure to efficiently prevent diseases caused by the mutated virus. Although several anti-viral drugs are available, these require a prescription and are known to have various side effects, such as nausea and diarrhea. Also, some of these anti-viral drugs are not recommended (or prohibited) for children or pregnant women. Therefore, the present invention addresses the need for rapidly and continuously available, cost-effective compositions and methods for treating or preventing viral infections. As these compositions comprise natural plant-derived or fungus-derived compounds, they are safe for long-term use, at all ages, and do not require any prescription.

The terms “comprises”, “comprising”, “include” and “including” as used herein refer to the presence of stated features, integers, components, or steps, but do not preclude the presence or addition of one or more other features, integers, components, steps, or groups thereof.

The term “respiratory virus (RV)” as used herein refers to a submicroscopic infectious agent that replicates inside the living cells of an organism (a host) and which affects the upper respiratory tract and/or the lower respiratory tract. The term encompasses both single-stranded and double-stranded DNA- and RNA-viruses. Non-limiting examples of RVs include adenovirus, bocavirus, coronavirus, enterovirus, influenza virus, parainfluenza virus, parvovirus, pneumovirus, respiratory syncytial virus (RSV), and rhinovirus.

Accordingly, diseases, disorders or pathological conditions caused by viral respiratory infections that can be treated or prevented according to the invention include, but are not limited to, viral infections that affect the upper and/or lower respiratory tracts, common cold, seasonal cold, influenza (flu), severe acute respiratory syndrome (SARS), coronavirus disease, pharyngitis, tonsillitis, laryngitis, sinusitis, bronchitis, bronchiolitis, viral pneumonia, acute otitis media, and croup (laryngotracheobronchitis).

The term “upper respiratory tract” as used herein refers to the parts of the respiratory tract that are higher on the body, i.e., nose, sinuses, pharynx, and larynx. Diseases and conditions caused by viral infections that affect the upper respiratory tract commonly include nasal obstruction, sore throat, tonsillitis, pharyngitis, laryngitis, sinusitis, otitis media, and common cold.

The term “lower respiratory tract” as used herein refers to the parts of the respiratory tract that are below the larynx, i.e., the trachea and lungs. Diseases and conditions caused by viral infections that affect the lower respiratory tract commonly include bronchitis, bronchiolitis, cough, and viral pneumonia.

It should be noted that some of the viral-mediated diseases mentioned above, such as influenza, may affect both the upper and lower respiratory tracts.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered.

In one embodiment, the subject is a mammal. In a specific embodiment, the subject is a human.

The term “patient” refers to a subject afflicted with a disease, disorder, or pathological condition.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, a treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prevent” or “preventing” refers to precluding, averting, obviating, forestalling, stopping, or hindering a disease, disorder, or pathological condition from happening, especially by advance action.

In one aspect, the present invention provides a composition comprising one or more plant-derived or fungus-derived compound.

According to the invention, the one or more plant- or fungus-derived compound is selected from the compounds listed in Table 1.

TABLE 1
List of plant- or fungus-derived compounds of the invention

| Name of compound | Chemical name | CAS No. |
|---|---|---|
| D-Limonene | 4R)-1-methyl-4-(prop-1-en-2- | 5989-27-5 |
| | yl)cyclohex-1-ene | |
| Menthol | 5-methyl-2-(propan-2-yl) | 1490-04-6 |
| | cyclohexan-1-ol | |
| Linalyl anthranilate | 1,6-Octadien-3-ol,3,7-dimethyl-, | 7149-26-0 |
| (linalyl 2- | 2-aminobenzoate | |
| aminobenzoate) | | |
| Zingiberene | (S-(R*,S*))-5-(1,5- | 495-60-3 |
| | Dimethylhexen-4-yl)-2-methyl- | |
| | 1,3-cyclohexa-1,3-dien | |
| p-Menthone (trans- | (2R,5S)-5-methyl-2-(propan-2- | 89-80-5 |
| menthone) | yl)cyclohexan-1-one | |
| Linalool | 3,7-dimethylocta-1,6-dien-3-ol | 78-70-6 |
| p-Menthan-3-one | (2S,5R)-5-methyl-2-(propan-2- | 10458-14-7 |
| | yl)cyclohexan-1-one | |
| 1,8-Cineole | 1,3,3-trimethyl-2- | 470-82-6 |
| | oxabicyclo[2.2.2]octane | |
| Tumerone | 2-Methyl-6-(4-methyl-1,3- | 180315-67-7 |
| (α-turmerone) | cyclohexadien-1-yl)-2-hepten- | |
| | 4-one | |
| Ar-tumerone | 2-methyl-6-(4-methylphenyl)-2- | 1000292-71-0 |
| | hepten-4-one | |
| (−)-β- | 3-[(2S)-6-methylhept-5-en-2-yl]- | 20307-83-9 |
| Sesquiphellandrene | 6-methylidenecyclohexene | |
| α-Curcurmene | 1-methyl-4-(6-methylhept-5-en- | 644-30-4 |
| | 2-yl)benzene | |
| (1S)-(−)-Camphor | 1,7,7-trimethylbicyclo[2.2.1]-2- | 464-48-2 |
| | heptanone | |
| Curlone | (6S)-2-methyl-6-[(1S)-4- | 87440-60-6 |
| | methylidenecyclohex-2-en-1-yl] | |
| | hept-2-en-4-one | |
| 2-β-Pinene | (1S,5S)-6,6-dimethyl-2- | 127-91-3 |
| | methylidenebicyclo[3.1.1]heptane | |
| β-Bisabolene | 6-methyl-2-(4-methyl-3- | 495-61-4 |
| | cyclohexen-1-yl)-1,5-heptadiene | |
| α-Pinene | 2,6,6-trimethylbicyclo[3.1.1] | 80-56-8 |
| | hept-2-ene | |
| γ-Terpinene | 1-methyl-4-(propan-2-yl) | 99-85-4 |
| | cyclohexa-1,4-diene | |
| Camphene | 2,2-dimethyl-3- | 79-92-5 |
| | methylidenebicyclo[2.2.1]heptane | |
| α-Farnesene | (6E)-7,11-dimethyl-3- | 502-61-4 |
| | methylidenedodeca-1,6,10-triene | |
| Terpinene-4-ol | 4-methyl-1-(propan-2-yl) | 562-74-3 |
| | cyclohex-3-en-1-ol | |
| γ-Muurolene | (1R,4aR,8aS)-7-methyl-4 | 30021-74-0 |
| | -methylidene-1-propan-2-yl- | |
| | 2,3,4a,5,6,8a-hexahydro-1H- | |
| | naphthalene | |
| Trans-Caryophyllene | (1R,4E,9S)-4,11,11-trimethyl-8- | 87-44-5 |
| (β-caryophyllene) | methylidenebicyclo[7.2.0] | |
| | undec-4-ene | |
| (L)-α-Terpineol | 2-(4-methylcyclohex-3-en-1-yl) | 10482-56-1 |
| | propan-2-ol | |
| (1R)-p-Mentha-3-ene | 1-isopropyl-4a- | 619-52-3 |
| | methylcyclohexene | |
| β-Ocimene | (E)-3,7-dimethylocta-1,3,6-triene | 3779-61-1 |
| (ocimene (E)) | | |
| β-Myrcene | 7-methyl-3-methylideneocta- | 123-35-3 |
| | 1,6-diene | |
| (±)-Lavandulol acetate | (±)-2-isopropenyl-5-methyl-4- | 1000352-62-6 |
| (lavandulyl acetate) | hexen-1-yl acetate | |
| o-Cymol (o-cymene) | 1-methyl-4-(propan-2-yl)benzene | 527-84-4 |
| (+)(−)Trans-Carane | bicyclo[4.1.0]heptane, | 18968-23-5 |
| | 3,7,7-trimethyl-,(1α,3α,6α)- | |
| Ocimene | cis-3,7-dimethyl-1,3,6-octatriene | 3338-55-4 |
| E-Citral (β-citral) | (2E)-3,7-dimethylocta-2,6-dienal | 141-27-5 |
| Alloaromadendrene | (1aR,7S,7bS)-1,1,7-trimethyl-4- | 25246-27-9 |
| | methylidene-2,3,4a,5,6,7,7a,7b- | |
| | octahydro-1aH-cyclopropa[e] | |
| | azulene | |

The present invention also encompasses compositions comprising any combination of the compounds listed in Table 1, for example, a combination comprising at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, or at least thirty-three of the compounds listed in Table 1.

According to one embodiment of the invention, the composition comprises the one or more plant- or fungus-derived compounds listed in Table 1 at the concentration ranges specified in Table 2.

TABLE 2
Concentration ranges of plant- or fungus-derived compounds in the
composition

| Name of compound | Concentration range (ppm) |
|---|---|
| D-Limonene | 6828-8346 |
| Menthol | 4946-6046 |
| Linalyl anthranilate (linalyl 2- | 3898-4766 |
| aminobenzoate) | |
| Zingiberene | 3571-4365 |
| p-Menthone (trans-menthone) | 2847-3481 |
| Linalool | 2592-3168 |
| p-Menthan-3-one | 2199-2689 |
| 1,8-Cineole | 2044-2500 |
| Tumerone (α-turmerone) | 1998-2442 |
| Ar-tumerone | 1937-2369 |
| (−)-β-Sesquiphellandrene | 1586-1940 |
| α-Curcurmene | 1263-1545 |
| (1S)-(−)-Camphor | 1095-1339 |
| Curlone | 1080-1322 |
| 2-β-Pinene | 1071-1309 |
| β-Bisabolene | 874-1070 |
| α-Pinene | 856-1048 |
| γ-Terpinene | 693-847 |
| Camphene | 683-835 |
| α-Farnesene | 655-801 |
| Terpinene-4-ol | 453-555 |
| γ-Muurolene | 440-538 |
| Trans-Caryophyllene (β-caryophyllene) | 360-440 |
| (L)-α-Terpineol | 355-435 |
| (1R)-p-Mentha-3-ene | 234-286 |
| β-Ocimene (ocimene (E)) | 215-263 |
| β-Myrcene | 201-247 |
| (±)-Lavandulol acetate (lavandulyl acetate) | 187-229 |
| o-Cymol (o-cymene) | 178-218 |
| (+)(−)Trans-Carane | 140-172 |
| Ocimene | 117-143 |
| E-Citral (β-citral) | 102-126 |
| Alloaromadendrene | 89-109 |

The present invention also encompasses compositions comprising one or more of the compounds listed in Table 1 at the concentration range according to Table 2 and additional compounds or active ingredients (other than those mentioned in Table 1).

According to a specific embodiment of the invention, the composition comprises the one or more plant- or fungus-derived compounds listed in Table 1 at the concentrations specified in Table 3.

TABLE 3
Concentrations of plant- or fungus-derived compounds in the composition

| Name of compound | Concentration (ppm) |
|---|---|
| D-Limonene | 7587 |
| Menthol | 5496 |
| Linalyl anthranilate (linalyl 2-aminobenzoate) | 4332 |
| Zingiberene | 3968 |
| p-Menthone (trans-menthone) | 3164 |
| Linalool | 2880 |
| p-Menthan-3-one | 2444 |
| 1,8-Cineole | 2272 |
| Tumerone (α-turmerone) | 2220 |
| Ar-tumerone | 2153 |
| (−)-β-Sesquiphellandrene | 1763 |
| α-Curcurmene | 1404 |
| (1S)-(−)-Camphor | 1217 |
| Curlone | 1201 |
| 2-β-Pinene | 1190 |
| β-Bisabolene | 972 |
| α-Pinene | 952 |
| γ-Terpinene | 770 |
| Camphene | 759 |
| α-Farnesene | 728 |
| Terpinene-4-ol | 504 |
| γ-Muurolene | 489 |
| Trans-Caryophyllene (β-caryophyllene) | 400 |
| (L)-α-Terpineol | 395 |
| (1R)-p-Mentha-3-ene | 260 |
| β-Ocimene (ocimene (E)) | 239 |
| β-Myrcene | 224 |
| (±)-Lavandulol acetate (lavandulyl acetate) | 208 |
| o-Cymol (o-cymene) | 198 |
| (+)(−)Trans-Carane | 156 |
| Ocimene | 130 |
| E-Citral (β-citral) | 114 |
| Alloaromadendrene | 99 |

According to a specific embodiment of the invention, the composition comprises D-limonene at a concentration range of 6828-8346 ppm, menthol at a concentration range of 4946-6046 ppm, and linalyl anthranilate at a concentration range of 3898-4766 ppm.

According to one embodiment of the invention, the plant- or fungus-derived compound is purchased as a commercially available product.

According to another embodiment of the invention the compounds of the invention are freshly extracted from plants or edible fungi. Extraction of the compounds from plants or edible fungi comprises obtaining an extract or essential oil of a plant or fungus using routine methods for extracting materials from natural sources manually or by the use of machines, such as solvent extraction (e.g., alcohol extraction), steam distillation (SD), hydrodistillation (HD), steeping (infusion), accelerated solvent extraction (ASE), maceration, percolation, reflux extraction, decoction, Soxhlet extraction, supercritical fluid (e.g., supercritical CO₂) extraction (SFE), simultaneous distillation solvent extraction (SDE), pressurized liquid extraction (PLE), microwave-assisted extraction (MAE), ultrasound-assisted extraction (UAE, sonication), microwave-assisted hydro-distillation (MWHD), purge and trap, solid phase microextraction (SPME), pulsed electric field (PEF) extraction, enzyme-assisted extraction (EAE), expression (for example, using a ram press), slow-folding (sfumatura), resin tapping, wax embedding, and cold pressing.

Recovery of a compound from a plant or edible fungus can further comprise separation or purification of the compound from the extract or essential oil. Said separation or purification can be carried out using routine methods, such as chromatography, in particular column chromatography. Non-limiting examples of chromatography techniques for purifying the desired compounds from the extract include adsorption column chromatography, partition chromatography (PC), membrane filtration (MF), gel filtration chromatography (GFC), ion-exchange chromatography (IEC), molecular distillation (MD), gas chromatography (GC), supercritical fluid chromatography (SFC), molecular imprinted technology, simulated moving bed (SMB) chromatography, and multi-dimensional chromatographic separation.

The term “extract” as used herein refers to a solution made by obtaining desirable substances from a plant without changing their qualities. Usually, the substances of the plant (or fungus) are diluted in a solvent (e.g., water, oil, alcohol).

The term “essential oil” as used herein refers to a liquid containing undiluted volatile chemical compounds of a plant or edible fungus.

According to an embodiment of the invention, each compound is derived from one or more plants and/or edible fungi selected from black elderberry (Sambucus nigra), turmeric (Curcuma longa), cat's claw (uña de gato, Uncaria tomentosa), female ginseng (Angelica sinensis), fennel (Foeniculum vulgare), licorice (Glycyrrhiza glabra), bitter orange (Seville orange, Citrus x aurantium), purslane (Portulaca oleracea), greater galangal (Alpinia galangal), myrrh (Commiphora myrrha), saffron (autumn crocus, Crocus sativus), flowering thick-stemmed sugar cane (Saccharum officinarum), ginger (Zingiber Officinale), lavender (Lavandula angustifolia), mint (peppermint, Mentha x piperita), rosemary (Rosmarinus Officinalis), lemon (Citrus x limon), reishi (lingzhi, Ganoderma lucidum), and Agaricus hortensis (also known as Agaricus bisporus; common mushroom in immature state, or portobello mushroom in mature state), or any combination thereof.

In a further embodiment of the invention, the composition comprises at least one entire extract or essential oil derived from one or more of the above-mentioned plants and/or fungi, such that one or more of the compounds listed in Table 1 are present in said composition at a concentration within the range specified in Table 2. The at least one extract or essential oil may be purchased as a commercially available product or freshly obtained from the plant or fungus.

In a specific embodiment of the invention, the composition comprises a combination of the extracts and essential oils derived from the plant species and fungi species listed above. An example of such a combination is a combination of extracts derived from cat's claw, reishi, black elderberry, turmeric, Agaricus hortensis, female ginseng, fennel, licorice, bitter orange, greater galangal, myrrh, saffron, purslane and flowering thick-stemmed sugar cane, and essential oils derived from turmeric, mint, ginger, lavender, lemon and rosemary, such that one or more of the compounds listed in Table 1 are present in said composition at a concentration within the range specified in Table 2.

The compositions according to the present invention may be in the form of tablets, pills, capsules, cachets, lozenges, granules, semisolids, powders, sustained release formulation, solutions, suspensions, emulsions, elixirs, aerosols or any other appropriate compositions. In addition, the compositions according to the invention may further include acceptable excipients (e.g., fillers (diluents), disintegrants, binders, glidants, and lubricants), colorants, sweeteners, flavorants, and/or preservatives.

According to one embodiment of the invention, the compositions of the invention are formulated as dietary supplements or as nutritional supplements.

As used herein, the term “dietary supplement” refers to a small amount of a compound, composition or combination of compounds for supplementation of a human or animal diet, wherein the dietary supplement is packaged in single or multiple dose units. Dietary supplements do not provide significant amounts of calories but may contain other micronutrients (e.g., vitamins or minerals).

The dietary supplements according to the present invention may be administered in any form (e.g., solution, pill, powder and food product).

The dietary supplements may further comprise one or more inert ingredients. For example, the dietary supplements may also contain vitamins, minerals, enhancers, colorants, sweeteners, flavorants, preservatives and/or inert ingredients.

In one embodiment of the invention, the dietary supplements are provided in liquid form to be used by adding to a drink (e.g., water, milk, juice, a carbonated drink, a hot drink or liquor), solid food (such as a slice of bread) or semi-solid food (such as a pudding, topping, sauce, puree, cooked cereal, or salad dressing.

The term “nutritional supplement” as used herein refers to a combination of a dietary supplement and a source of calories. In some embodiments of the invention, nutritional supplements are meal replacements or meal supplements (such as, a nutrient or energy bar or a nutrient beverage, shake or concentrate).

In yet another aspect, the present invention relates to a method of treating or preventing a viral infection or disease, disorder or condition caused by a viral infection in a subject, the method comprising administering to a subject a composition as described above.

According to one embodiment of the invention, the compositions disclosed herein are orally administrated to the subject.

In some embodiments of the invention, the composition is administered therapeutically; that is, administered to treat an existing disease, disorder or condition. In other embodiments, the composition is administered prophylactically; that is, administered for prevention of a disease, disorder or condition.

In one embodiment of the invention, the compositions are administered concurrently with, but not limited to, a vaccination and/or anti-viral medications (including virustatic and virucidal drugs).

As mentioned above, the term “treatment” encompasses the relief of symptoms of a disease. Accordingly, the present invention also encompasses a method for alleviating symptoms of a disease, disorder or condition caused by a viral infection by administering the compositions of the invention as hereinbefore described.

The term “symptom(s)” as used herein, refers to common signs or indications that a subject is suffering from a specific condition or disease. For example, symptoms associated with a viral respiratory infection include, but are not limited to, rhinorrhea, scratchy/sore throat, fever, headache, sneeze, cough, exhaustion/fatigue, muscular aches, sore joints, irritated/watery eyes, malaise, nausea and/or vomiting, shaking chills, chest pain, inflamed respiratory mucous membranes, substernal burning, stridor, hoarse voice, loss of smell and/or taste, conjunctivitis, skin rash, discoloration of the fingers or toes, difficulty breathing or shortness of breath, chest pain or pressure, loss of speech or movement, exacerbation of chronic obstructive pulmonary diseases (COPD), and exacerbation of asthma.

Accordingly, the compositions of the invention are effective in reducing the severity and/or duration of the symptoms of diseases or conditions caused by a viral infection, in enhancing the recovery of patients from the infection, and in reducing long-term morbidity, sequelae or complications that result from the viral infection.

The treatment and/or prevention of viral infections, as well as diseases, disorders and conditions caused by viral infection and their symptoms, may involve the manifestation of antiviral effects possessed by the plant- or fungus-derived compounds which are present in the compositions of the invention. These antiviral effects may include inhibition of viral attachment and/or replication, for example, by enhancing the subject's immune response towards the virus, as well as modulating cellular defense and repair mechanisms. Examples of anti-viral effects include, but are not limited to, promoting the expression of proteins (such as proteases, defensins) that may prevent viral attachment and/or replication, regulating the expression/secretion of immunomodulatory cytokines (for example, increasing the expression of interferon-α/β, IL-1β and IL-18), activation of inflammatory cellular components (e.g., inflammasomes), modulating the activity of immune cells (such as macrophages and T helper cells), modulating the production of antibodies (such as specific secretory immunoglobulin A).

In one embodiment of the invention, the compositions disclosed herein are in liquid form and are administered to a subject dropwise directly on the subject's tongue. According to another embodiment of the invention, in order to increase the absorption of the active materials in the composition and consequently elevating their efficacy, the compositions of the invention are diluted into a drink (e.g., water, milk, juice, a carbonated drink, a hot drink and liquor) and subsequently consumed by the subject. In a specific embodiment of the invention, the volume of the drink ranges between 5 ml to 250 ml, particularly between 20 ml to 250 ml, and more particularly, between 50 ml to 95 ml. Alternatively, the compositions of the invention can be diluted in about a tenth of a cup to about one full cup, the cup typically representing a volume of between 200 to 250 ml. In yet another embodiment, the compositions of the invention are provided in solid or semi-solid food, for example, as an addition to a stew or dripped onto a slice of bread. The drink, solid food or semi-solid food is then consumed by the subject.

Liquid forms of the compositions according to the invention include the forms of solutions, oils, suspensions, emulsions, and elixirs. The liquid forms of the composition also include solid forms (e.g., tablets, powders, etc.) that were transformed into liquids, such as by melting or dissolving in liquids, as long as the liquid form contains one or more of the compounds listed in Table 1 at a concentration range as specified in Table 2.

The compositions of the invention can be administered daily, at an amount of between 1 drop and up to 100 drops per day (about 0.03 ml to about 6 ml). The total dose per day can be administered at once or divided into small amounts that are administered several times at the same day.

In a specific embodiment of the invention, the composition is administered to the subject at an amount of about 4 to about 15 drops per day. This amount is recommended mainly to a healthy subject (i.e., a subject not infected by a virus or a subject that has no immune system deficiencies) in order to prevent a viral infection, and considered by the inventors as an amount of the composition that is suitable to maintain the immune system of the subject deficiency-free or to further strengthen the subject's immune response against an invading pathogen (e.g., a virus).

In another specific embodiment of the invention, the composition is administered to a subject infected by a virus or displays symptoms of a disease, disorder or condition caused by a viral infection or diagnosed with said disease, disorder or condition at an amount of about 20 drops to about 100 drops per day.

In yet another specific embodiment of the invention, the compositions of the invention are administered to a patient diagnosed with a disease, disorder or pathological condition caused by a viral infection or shows symptoms of said disease, disorder or condition, an amount of about 70-100 drops per day during the first 1-3 days after the diagnosis was made or after the onset of the symptoms, followed by an amount of about 20-40 drops per day for additional 1-14 days, or until the patient has recovered (no longer infected by the virus), or until the symptoms have substantially subsided or completely disappeared. Once the patient has recovered, or the symptoms have substantially subsided or completely disappeared, the compositions of the invention may be administered to the subject at a “preventive amount”, for example, 4-15 drops per day.

As would be appreciated by a person of skills in the art, maintaining a healthy (deficiency-free) immune system in a subject or strengthening a subject's immune system may include facilitating and/or enhancing an immune response of the subject against an invading pathogen (e.g., a virus).

In a further aspect, the present invention provides compositions comprising one or more plant- or fungus-derived compound, as disclosed hereinabove, for use in treating or preventing a viral infection or disease, disorder or condition caused by a viral infection.

As mentioned above, the compositions provided herein can also be used to alleviate symptoms of a diseases or condition caused by a viral infection.

### Example 1

### Anti-Viral Activity of the Composition

The potential anti-viral activity of the compositions of the invention evaluated in a cellular model of SARS-CoV-2.

Methods and Materials

VERO E6 (ATCC® CRL-1586) was obtained from BEI Resources. All experiments were carried out in a Biosafety level 3 laboratory and conducted under appropriate conditions. Vero E6 cells were cultured in Minimum Essential Media (MEM) supplemented with 2% fetal bovine serum, penicillin/streptomycin (100 μg/mL) and L-glutamine (2 mM). The cells were cultured at 37° C. under 5% CO2, seeded in 96-well flat bottom microplates at 5×10⁴ cells/ml and incubated overnight to reach sub-confluence. The cells were pretreated with a composition according to one embodiment of the invention or with vehicle (ethanol) at a concentration of 0.125% for 4 hours before being infected with SARS-CoV-2 using an MOI of 0.5 for 1 hour. Uninfected cells were used as a negative control (NC). After 48 hours, viral-induced cytotoxicity was evaluated using MTT (Invitrogen) based on the manufacturer's protocol and presented as relative absorbance (RA).

The composition according to the invention tested in the experiments is a composition comprising all the compounds listed on Table 1 at the concentrations specified in Table 3.

Results

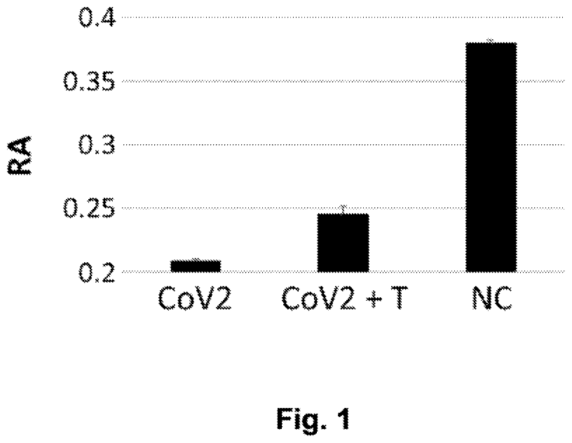

As shown in FIG. 1, VERO E6 cells pretreated with vehicle and infected with SARS-CoV-2 (CoV2) showed high level of cytotoxicity compared to uninfected cells. However, pretreatment with the composition specified above (CoV2+T) reduced viral-induced cytotoxicity in the cells by over 20% compared to the untreated control (CoV2).

These results indicate that the composition of the invention has anti-viral activity and is successful in treating respiratory viral infections, such as COVID-19.

1-27. (canceled)

28. A method of treating or preventing a viral respiratory infection or disease, disorder or condition caused by a viral respiratory infection in a subject, comprising administering a composition comprising plant-derived or fungus-derived compounds to the subject, wherein the compounds are D-Limonene, menthol, and linalyl anthranilate, and wherein D-Limonene, menthol, and linalyl anthranilate are present in the composition at concentration ranges according to the following table:

| Name of compound | Concentration range (ppm) |
|---|---|
| D-Limonene | 6828-8346 |
| Menthol | 4946-6046 |
| Linalyl anthranilate | 3898-4766 |

29. The method according to claim 28, further comprising one or more plant-derived or fungus-derived compounds selected from zingiberene, p-menthone, linalool, p-menthan-3-one, 1,8-cineole, tumerone, Ar-tumerone, (−)-β-sesquiphellandrene, α-curcurmene, (1S)-(−)-camphor, curlone, 2-β-pinene, β-bisabolene, α-pinene, γ-terpinene, camphene, α-farnesene, terpinene-4-ol, γ-muurolene, trans-caryophyllene, (L)-α-terpineol, (1R)-p-mentha-3-ene, β-ocimene, β-myrcene, (±)-lavandulol acetate, o-cymol, (+)(−)trans-carane, ocimene, β-citral and alloaromadendrene, or any combination thereof.

30. The method according to claim 29, wherein the one or more plant-derived or fungus derived compounds are present in the composition at concentration ranges according to the following table:

| Name of compound | Concentration range (ppm) |
|---|---|
| Zingiberene | 3571-4365 |
| p-Menthone | 2847-3481 |
| Linalool | 2592-3168 |
| p-Menthan-3-one | 2199-2689 |
| 1,8-Cineole | 2044-2500 |
| Tumerone | 1998-2442 |
| Ar-tumerone | 1937-2369 |
| (−)-β-Sesquiphellandrene | 1586-1940 |
| α-Curcurmene | 1263-1545 |
| (1S)-(−)-Camphor | 1095-1339 |
| Curlone | 1080-1322 |
| 2-β-Pinene | 1071-1309 |
| β-Bisabolene | 874-1070 |
| α-Pinene | 856-1048 |
| γ-Terpinene | 693-847 |
| Camphene | 683-835 |
| α-Farnesene | 655-801 |
| Terpinene-4-ol | 453-555 |
| γ-Muurolene | 440-538 |
| Trans-Caryophyllene | 360-440 |
| (L)-α-Terpineol | 355-435 |
| (1R)-p-Mentha-3-ene | 234-286 |
| β-Ocimene | 215-263 |
| β-Myrcene | 201-247 |
| (±)-Lavandulol acetate | 187-229 |
| o-Cymol | 178-218 |
| (+)(−)Trans-Carane | 140-172 |
| Ocimene | 117-143 |
| β-citral | 102-126 |
| Alloaromadendrene | 89-109 |

31. The method according to claim 28, wherein the compounds are derived from one or more plants and/or edible fungi selected from black elderberry, turmeric, cat's claw, female ginseng, fennel, licorice, bitter orange, purslane, greater galangal, myrrh, saffron, flowering thick-stemmed sugar cane, ginger, lavender, mint, rosemary, lemon, reishi, Agaricus hortensis, or any combination thereof.

32. The method according to claim 31, wherein the composition comprises at least one extract or essential oil derived from one or more of said plants and/or edible fungi.

33. The method according to claim 31, wherein the composition comprises extracts derived from cat's claw, reishi, black elderberry, turmeric, Agaricus hortensis, female ginseng, fennel, licorice, bitter orange, greater galangal, myrrh, saffron, purslane and flowering thick-stemmed sugar cane, and essential oils derived from turmeric, mint, ginger, lavender, lemon and rosemary.

34. The method according to claim 28, wherein the composition is in the form of a tablet, pill, capsule, cachet, lozenge, granule, semisolid, powder, sustained release formulation, solution, suspension, emulsion, elixir, or aerosol.

35. The method according to claim 28, wherein the composition is formulated as a dietary supplement or as a nutritional supplement.

36. The method according to claim 28, wherein the composition is administered to the subject concurrently with a vaccination and/or an anti-viral medication.

37. The method according to claim 28, wherein the composition is administered orally to the subject.

38. The method according to claim 37, wherein the composition is administered to the subject by adding said composition to a drink, solid food or semi-solid food to be consumed by the subject.

39. The method according to claim 37, wherein the composition is in liquid form and is administered to the subject at an amount of between 1 to 100 drops per day.

40. The method according to claim 39, wherein preventing a viral respiratory infection or disease, disorder or condition caused by a viral respiratory infection comprises administering the composition to the subject at an amount of 4-15 drops per day.

41. The method according to claim 39, wherein treating a viral respiratory infection or a disease, disorder or condition caused by a viral respiratory infection or alleviating symptoms of a disease, disorder or condition caused by a viral respiratory infection in the subject comprises administering the composition to the subject at an amount of 20-100 drops per day.

42. The method according to claim 41, wherein treating a viral respiratory infection or a disease, disorder or condition caused by a viral respiratory infection or alleviating symptoms of a disease, disorder or condition caused by a viral respiratory infection in the subject comprises administering the composition to the subject at an amount of 70-100 drops per day during the first 1-3 days after the diagnosis of the disease, disorder or condition was made or after the onset of the symptoms, followed by an amount of 20-40 drops per day for additional 1-14 days, or until the patient has recovered, or until the symptoms have subsided or completely disappeared.

43. A method of alleviating symptoms of a disease, disorder or condition caused by a viral respiratory infection in a subject, comprising administering a composition comprising plant-derived or fungus-derived compounds to the subject, wherein the compounds are D-Limonene, menthol, and linalyl anthranilate, and wherein D-Limonene, menthol, and linalyl anthranilate are present in the composition at concentration ranges according to the following table:

| Name of compound | Concentration range (ppm) |
|---|---|
| D-Limonene | 6828-8346 |
| Menthol | 4946-6046 |
| Linalyl anthranilate | 3898-4766 |
